(19) United States
(12) Patent Application Publication
(10) Pub. No.: US 2023/0088977 A1
(43) Pub. Date: Mar. 23, 2023

(54) GUIDE CATHETER EXTENSION SYSTEM FOR REVERSE CONTROLLED ANTEGRADE/RETROGRADE TRACKING & THROMBUS REMOVAL PROCEDURES

(71) Applicant: CROSSLINER INC., KALAMAZOO, MI (US)

(72) Inventors: TIM A. FISCHELL, KALAMAZOO, MI (US); FRANK S. SALTIEL, WILLOWBROOK, IL (US); AARON GRANTHAM, OVERLAND PARK, KS (US)

(21) Appl. No.: 18/051,221

(22) Filed: Oct. 31, 2022

Related U.S. Application Data
(63) Continuation-in-part of application No. 16/793,120, filed on Feb. 18, 2020.
Application No. 16/793,120 is a continuation-in-part of application No. 16/132,878, filed on Sep. 17, 2018.
Application No. 16/132,878 is a continuation-in-part of application No. 15/899,603, filed on Feb. 20, 2018, now Pat. No. 11,491,313.

(51) Int. Cl. A61M 25/10; A61F 2/958

(52) U.S. Cl. CPC A61M 25/104; A61F 2/958

(57) ABSTRACT

The guide catheter extension system for various intravascular procedures, including the reverse CART procedure, the thrombus removal, etc., has an enhanced ”capturing” capability. It is configured with a plastically expandable scaffold member forming an expandable “funnel-like” distal opening, and, once it has been advanced into the subintimal space, provides an enhanced capability of catching the retrograde wire or a thrombus, as required by the procedure. A balloon delivered to the target location in the blood vessel, by being inflated, opens the scaffold member to enhance the delivery of the retrograde wire or the thrombus into the guide catheter extension. When the guide catheter extension is no longer needed, the flared guide extension can be easily compressed and collapsed as it is drawn in the guiding catheter. For benefits of the thrombus removal, the balloon may be formed from a material loaded with a radiopaque material and prefabricated with micro pores. A thrombolytic agent can be delivered to the thrombus before the thrombus is conveniently captured in the expanded distal opening of the scaffold member and removed from the blood vessel by aspiration. The outer or inner catheter may be configured with a distal curved portion to enhance a rotational capability for displacement between the right and left pulmonary arteries.

### INCORPORATION BY REFERENCE

U.S. Pat. Applications Serial No. 15/899,603 filed 20 Feb. 2018; Serial No. 16/132,878 filed 17 Sep. 2018; and Serial No. 16/793,120, filed 18 Feb. 2020, currently pending, are hereby incorporated by reference in their entirety.

### FIELD OF THE INVENTION

The present invention is directed to medical devices, and particularly, to devices designed for intravascular surgical procedures, such as, for example, the reverse Controlled Antegrade and Retrograde Tracking (CART) procedure, thrombus removal, and other fast and efficient procedures in various blood vessels in a patient’s body.

The present invention is also directed to an improved guide catheter extension system for a safe and simple delivery and access to undesirable formations in various blood vessels of the patient’s body and therapeutic treatment.

The present invention is further directed to an enhanced reverse CART procedure by using an antegrade guide catheter extension system which has an enhanced ability to “capture” a retrograde wire when inserted in the shared subintimal space, thus decreasing the duration of and easing the reverse CART procedure.

The present invention addresses an efficient reverse CART technique for a chronic total occlusion (CTO) intervention in a blood vessel, where a retrograde guidewire may be entered from a collateral or a potentially degenerated bypass graft into a target blood vessel and creates an intentional subintimal dissection in the subintimal space in the target blood vessel, while a modified antegrade guide catheter extension system is entered into the subintimal space from the antegrade approach to capture the retrograde wire in the most efficient and expedited manner.

In addition, the present invention provides a modified guide catheter extension system designed to overcome shortcomings in the routine of capturing the retrograde wire within the shared subintimal space in the blood vessel of interest, where the modified guide catheter extension system has a radially expandable distal opening at the distal end configured in a flared configuration and capable of deforming and expanding to form a “funnel-like” distal opening at the distal end of the guide catheter extension system once it has been advanced into the subintimal space, thus providing favorable configuration of the distal opening for catching the retrograde wire therein.

The present invention also provides a guide catheter extension system configured with a radially expanding scaffold member at the distal end, which forms a distal opening controllably expanding for the increased capturing capability at the distal end.

Furthermore, the present invention is focused on an improved guide catheter extension system integrating an antegrade balloon catheter which incorporates a balloon in operative coupling with a deformable shape memory scaffold structure, where the balloon is inflated to flare the deformable shape memory scaffold structure to expand the distal opening and, thus, to enhance the delivery of the retrograde wire into the distal end of the guide catheter extension system, wherein, subsequent to the inner balloon catheter is used to “flaring” of the distal end of the guide catheter extension system, the balloon is deflated and removed from the blood vessel to permit the entry of the retrograde wire / retrograde microcatheter / retrograde catheter into the guide catheter extension system, and wherein, once the guide catheter extension is no longer needed, the flared scaffold structure can be plastically collapsed as it is withdrawn through the guide catheter.

In addition, the present invention addresses a thrombus removal procedure performed with an expandable catheter extension system which includes an outer thrombus removing catheter and an inner balloon catheter, where the outer thrombus removing catheter is configured with a deformable shape memory scaffold structure at the distal end which is overlapped with a balloon on the inner catheter, where the balloon on the inner balloon catheter is inflated when delivered into the blood vessel of interest to flare the deformable scaffold structure to expand the distal opening and, thus, to ease the insertion of the thrombus into the distal opening at the distal end of the outer thrombus removing catheter. Subsequent to the inner balloon catheter is used to “flaring” of the distal end of the guide catheter extension system, the balloon is deflated and removed from the blood vessel to permit the entry of the thrombus into the outer thrombus removing catheter. The thrombus is removed by aspiration of the thrombus along the outer catheter lumen with the help of a vacuum system/syringe coupled to the proximal end of the outer thrombus removing catheter. Once the guide catheter extension is no longer needed, the flared scaffold structure can be plastically collapsed as it is withdrawn through a sheath or guide catheter.

The present invention also addresses a guide catheter extension system capable of rotational displacement between the right and left pulmonary arteries, especially beneficial for the thrombectomy procedure, where the outer catheter or the inner catheter is configured with a pre-shaped or deflectable curve at the distal end of the outer or inner catheter.

In addition, the present invention addresses a guide catheter extension system capable of the thrombectomy procedure enhanced with delivery of a thrombolytic agent to a clot within a pulmonary or coronary artery prior to the clot removal, where the thrombolytic agent is delivered to the clot through an inflatable balloon fabricated from a radiopaque material (such as tungsten, and/or barium, gold, etc. loaded material) with micro pores serving as the medicinal fluid delivery ports.

### BACKGROUND OF THE INVENTION

Percutaneous intervention (PCI) in peripheral and coronary arteries is a commonly performed procedure. Recently, advances in coronary interventions which include complex interventions to open chronic total occlusion’s (CTO’s), have led to requirements for novel guide extension catheters.

Reverse CART procedure is one of the most complex coronary interventional procedures recently performed for open chronic total occlusions. As presented in FIGS. 1A-1F, the reverse CART surgery is typically performed to treat chronic total occlusion (CTO) lesion(s) 10 formed within a blood vessel 12. The reverse CART procedure can treat blockages fully occluding a blood vessel as shown in FIG. 1A. The reverse CART procedure involves an initial purposeful dissection in proximity to the blockage created from an antegrade approach 14, as well as from a retrograde approach 16, to create a subintimal space 22 inside the blood vessel 12, as shown in FIG. 1B. Subsequently, an antegrade guidewire 18 is advanced from the proximal end 20 of the blood vessel 12 into the subintimal space 22 formed by the antegrade and retrograde dissections. The antegrade guide wire 18 is used for advancing an antegrade catheter (also referred to herein as a guide extension catheter) 19 carrying an antegrade balloon 26 to enter (in its deflated state) into the subintimal space 22. The antegrade catheter 19 typically has a micro - catheter 24 formed at the distal end of the antegrade catheter. The micro-catheter 24 slides over the antegrade guide wire 18 during displacement of the antegrade catheter 19 along the antegrade guide wire 18. The guide extension catheter may have a diameter which is approximately ⅓ of the blood vessel (artery) diameter.

Prior to or after the introduction of the retrograde wire 28 into the subintimal space 22, the antegrade balloon 26 arriving in the subintimal space 22, may be inflated to expand the subintimal space 22, as shown in FIG. 1C.

As depicted in FIG. 1D, a retrograde guide wire 28 is delivered in the subintimal space 22 (which is expanded in size by the inflated antegrade balloon 26).

As shown in FIG. 1E, an antegrade guide extension catheter 19 is used to “catch” a distal end 30 of the retrograde wire 28 into the distal opening 32 of the antegrade guide extension catheter 19.

Once the distal end 30 of the retrograde wire 28 is collected into the distal opening of the antegrade guide extension catheter 19, a retrograde microcatheter 34 is advanced over the retrograde guide wire 28 into the distal opening 32 of the antegrade guide extension catheter 19, as shown in FIG. 1F. At this point, the retrograde guide wire 28 is removed (the antegrade guide extension catheter 19 can also be removed from the blood vessel), and a long (approximately 350 cm) retrograde wire is advanced through the microcatheter 34 into the guide catheter and can subsequently be advanced further to exit through the antegrade access site.

The long retrograde wire can subsequently be used from the antegrade approach 14 to complete the coronary revascularization station with the angioplasty, typically followed by the coronary stenting, to reconstruct the blood vessel from the distal true lumen to the proximal true lumen on both sides of the CTO lesion 10.

One of the challenges in the typical reverse CART procedure is the capture of the retrograde wire 28 into the antegrade catheter 19 in the shared subintimal space 22. Specifically, it may be extremely difficult to locate the distal opening 32 of the antegrade catheter 19 with the distal end 30 of the retrograde wire 28. Multiple attempts with limited steering are typically performed during the procedure, and often wire exchanges to the retrograde system may be required to capture the retrograde wire into the antegrade catheter 19, thus resulting in the extended duration and undesired complexity of the reverse CART procedure.

It would therefore be highly desirable to overcome the difficulty of capturing the retrograde wire into the distal opening of the antegrade guide catheter tubular extension in the shared subintimal space during the reverse CART surgery to decrease the duration of the surgery and to ease the procedure performance.

### SUMMARY OF THE INVENTION

It is therefore an object of the present invention to improve the reverse CART procedure by using an antegrade guide catheter extension system which has an enhanced ability to “capture” a retrograde wire when inserted in the shared subintimal space, thus decreasing the duration and easing the reverse CART procedure.

It is another object of the present invention to provide an efficient reverse CART technique for a chronic total occlusion (CTO) intervention in a blood vessel, where a retrograde guidewire may be entered from a collateral or a potentially degenerated bypass graft into a target blood vessel and creates an intentional subintimal dissection in the subintimal space in the target blood vessel, while a modified antegrade guide catheter extension system is entered into the subintimal space from the antegrade approach to capture the retrograde wire in the most efficient and expedited manner.

It is an additional object of the present invention to provide a modified guide catheter extension system designed to overcome shortcomings in the routine of capturing the retrograde wire within the shared subintimal space in the blood vessel of interest, where the modified guide catheter extension system has a radially expandable distal opening at the distal end configured in a flared configuration and capable of deforming and expanding to form a “funnel-like” distal opening at the distal end of the guide catheter extension system once it has been advanced into the subintimal space, thus providing favorable configuration of the distal opening for catching the retrograde wire therein.

It is another object of the present invention to provide a guide catheter extension system configured with a radially expanding scaffold member at the distal end, which forms a distal opening controllably expanding for the increased capturing capability at the distal end.

It is a further object if the present invention to provide an improved guide catheter extension system integrating an antegrade balloon catheter which incorporates a balloon in operative coupling with a deformable scaffold structure, where the balloon is inflated to flare the deformable shape memory scaffold structure to expand the distal opening and, thus, to enhance the delivery of the retrograde wire into the distal end of the guide catheter extension system, wherein, subsequent to the inner balloon catheter is used to “flaring” of the distal end of the guide catheter extension system, the balloon is deflated and removed from the blood vessel to permit the entry of the retrograde wire / retrograde microcatheter / retrograde catheter into the guide catheter extension system, and wherein, once the guide catheter extension is no longer needed, the flared scaffold structure can be plastically collapsed as it is withdrawn through the guide catheter.

It is an additional object of the present invention to provide an expandable guide catheter extension system applicable to a thrombus removal procedure and an enhanced thrombus removal procedure where the expandable guide catheter extension system, or sheath system, includes an outer thrombus removing catheter and an inner balloon catheter, where the outer thrombus removing catheter is configured with a deformable scaffold structure at the distal end which is overlapped with a balloon on the inner catheter, where the balloon on the inner balloon catheter is inflated when delivered into the blood vessel of interest to flare the deformable scaffold structure to expand the distal opening and, thus, to enhance the collection of the thrombus into the distal opening at the distal end of the outer thrombus removing catheter and aspiration of the thrombus along the outer catheter lumen with the help of a vacuum system./syringe coupled to the proximal end of the outer thrombus removing catheter, wherein, subsequent to the inner balloon catheter is used to “flaring” of the distal end of the guide catheter extension system, the balloon is deflated and removed from the blood vessel to permit the entry of the thrombus into the outer thrombus removing catheter, and wherein, once the guide catheter extension, or sheath, is no longer needed, the flared scaffold structure can be plastically collapsed as it is withdrawn through the guide catheter or through the sheath at the groin.

In one aspect, the present invention constitutes an expandable guide catheter extension system for percutaneous intervention in peripheral and/or coronary arteries to open the chronic total occlusion (CTO) as performed by the reverse CART technique. The subject intravascular guide catheter extension system is configured for the reverse controlled antegrade and retrograde tracking (CART) procedure in a blood vessel of interest having chronic total occlusion leisure.

The subject system is configured with an outer member formed by a flexible substantially cylindrically contoured elongated outer sheath defining an outer sheath lumen having a proximal end and a distal end. The outer sheath extends between a middle portion and a distal portion of the intravascular guide catheter extension system. The outer sheath is configured with an outer tip (which may be a tapered outer tip) at the distal end of the outer sheath lumen.

A radially expandable scaffold member is positioned at the outer tip at the distal end of the outer sheath. The radially expendable scaffold member may be configured with a plastically deformable elongated member (wire) which is shaped into a zig-zag configuration to form a plurality of wing members. Each wing member extends longitudinally of the outer sheath, and the number of the wing members are disposed circumferentially along the walls of the outer sheath around a longitudinal axis of the outer sheath.

The radially expandable scaffold member can intermittently assume a closed configuration and an opened configuration. In the closed configuration, the wing members of the radially expandable scaffold member are arranged in a cylindrical tubular formation having a proximal opening and a distal opening with approximately equal diameters. While in the opened configuration, the scaffold member is plastically deformed to radially expand the distal ends of the wing members from one another to enlarge the distal opening formed by the distal ends of the wing members of the radially expandable scaffold member. The shape memory elongated member (wire) of the scaffold member maintains the opened configuration as required by the reverse CART procedure.

The subject system further includes an inner member which has an elongated body defining an internal channel extending along its longitudinal axis. The inner member extends internally along the outer sheath lumen of the outer sheath of the outer member in a controllable relationship with the outer sheath. The inner member has a tapered distal tip configured with a tapered delivery micro-catheter having an elongated body of a predetermined length. The tapered delivery micro-catheter can be displaced along the guide wire beyond the distal end of the outer sheath.

The inner member is configured with a balloon member having a distal section and a proximal section. The balloon member is attached at its proximal and distal sections to the tapered distal tip of the inner member. The proximal section of the balloon member extends internally of the radially expandable scaffold member at the distal end of the outer sheath.

An inflation lumen extends inside the inner member between the proximal section of the subject guide catheter extension system and the balloon member to provide a fluid passage between a balloon inflation system and the balloon member. The balloon member can assume intermittently an inflated configuration and deflated configuration. When the balloon member is controlled to assume the inflated configuration by actuating the balloon inflation system, the proximal section of the balloon member expands and causes the radially expandable scaffold member to assume its opened configuration.

During the reverse CART procedure, the subject intravascular guide catheter extension system is delivered in the blood vessel of interest from its one end, while an additional catheter is delivered into the blood vessel of interest from its another end. The additional catheter has a proximal end and a distal end, wherein the distal end of the additional catheter is received in the radially expandable scaffold member in its opened configuration through the distal opening defined by the wing members.

In some embodiments, the subject intravascular guide catheter extension system may be an antegrade guide catheter extension system, and the additional catheter may be a retrograde catheter.

The elongated member (wire) may be formed from materials which can create the plastically deformed distal end of the outer catheter, such as, for example, Nitinol, stainless steel, cobalt chromium, and/or other alloys and plastics and materials, and their combination, which allow plastic deformation properties.

The subject intravascular guide catheter extension system further comprises an elastic sheath disposed at the distal end of the outer sheath of the outer member. The elastic sheath may be formed with a tubularly shaped portion and tapered portion disposed in an encircling relationship with the outer tip of the distal end of the outer sheath and the proximal section of the balloon member. The wing members configured with the elongated member (wire) are embedded into the tubularly shaped portion of the elastic sheath.

Each of the wing members has a distal end and a proximal end. The distal ends of the plurality of wing members form the distal opening of the radially expandable scaffold member. The proximal ends of the plurality of wing members form the proximal opening of the radially expandable scaffold member. In the opened configuration of the radially expandable scaffold member, the distal ends of the wing members space apart from one another, thus increasing a diameter of the distal opening.

The subject intravascular guide catheter extension system further comprises an interconnection mechanism disposed in an operative coupling with the inner and outer members and controllably actuated to operate the guide catheter extension system in an engaged or disengaged modes of operation. The interconnection mechanism is configured to prevent a displacement of the inner member relative to the outer member.

In the engaged mode of operation, the inner and outer members of the guide catheter extension system are engaged for a controllable common displacement along the guide wire, and in the disengaged mode of operation, the inner and outer members are disengaged for retraction of the inner member from the outer member subsequent to the deflation of the balloon member. During and upon the retraction of the inner member from the outer member, the radially expandable scaffold member Maintains its opened configuration.

The subject intravascular guide catheter extension system operates in conjunction with a guide catheter insertable in the blood vessel of interest. The intravascular guide catheter extension system slidably extends within and along the guide catheter. When the outer member is retracted from the guide catheter, the plurality of wing members are plastically compressed inside the guide catheter to allow longitudinal motion of the radially expandable scaffold member inside the guide catheter.

The interconnection mechanism may be based on different principles, for example, it may be a friction-based unit interfacing an outer surface of the inner member and an inner surface of the outer sheath of the outer member to create a “stop-fit”, and/or it may be a snap-fit mechanism configured with at least one snap-fit post formed at the inner member and extending above its external surface.

Preferably, a flat wire helical coil member may be used to form at least a portion of respective walls of the outer sheath of the outer member, and/or the micro-catheter. The flat wire helical coil may be formed with a material comprising Nitinol, a radio-opaque material, and combination thereof.

For visualization of the reverse CART procedure, the present intravascular system may utilize radio-opaque markers attached to distal end of the outer sheath, a distal end of the micro-catheter, to the tapered distal tip of the inner member in proximity to the proximal and distal sections of the balloon member, as well as other location required by the procedure.

Preferably, a curved portion is pre-shaped at the distal portion of the intravascular guide catheter extension system. The curved portion may be prefabricated at the distal end of the outer sheath of said outer member or at the distal portion of inner member. The curved portion angularly deviates from a longitudinal axis of the outer sheath at its proximal end at an angle ranging between 30° and 90°.

The balloon member may be formed with a tungsten, barium, gold, or other radiopaque loaded balloon material fabricated with a plurality of micro pores. A medicinal fluid is delivered into the balloon member through the inflation lumen, so that the medicinal fluid exits from the balloon member through the plurality of micro pores when a pressure inside the balloon member is reached sufficient to expel the medicinal fluid from the balloon member.

In another aspect, the present invention constitutes a method for reverse controlled antegrade and retrograde tracking (CART) using a modified guide catheter extension system. The subject method includes the Step A for assembling a modified intravascular guide catheter extension system configured with:
an outer member formed by a flexible substantially cylindrically contoured elongated outer sheath defining an outer sheath lumen having a proximal end and a distal end,
a radially expandable scaffold member positioned at an outer tip at the distal end of the outer sheath. The radially expendable scaffold member may be configured with an elongated member (wire) which is shaped into a zigzag, tubular-slotted configuration to form a plurality of wing members. Each wing member extends longitudinally of the outer sheath, and the number of the wing members are disposed circumferentially along the walls of the outer sheath around a longitudinal axis of the outer sheath.

The radially expandable scaffold member can intermittently assume a closed configuration and an opened configuration. In the closed configuration, the wing members of the radially expandable scaffold member are arranged in a cylindrical tubular formation having a proximal opening and a distal opening with approximately equal diameters. In the opened configuration, the scaffold member is plastically deformed to radially expand the distal ends of the wing members from one another to enlarge the distal opening formed by the distal ends of the wing members of the radially expandable scaffold member. The elongated member (wire) of the scaffold member maintains the opened configuration as required by the reverse CART procedure.
an inner member which has an elongated body defining an internal channel extending along its longitudinal axis. The inner member extends internally along the outer sheath lumen of the outer sheath of the outer member in a controllable relationship with the outer sheath. The inner member has a tapered distal tip configured with a tapered delivery micro-catheter having an elongated body of a predetermined length. The tapered delivery micro-catheter can be displaced along the guide wire beyond the distal end of the outer sheath.
a balloon member having a distal section and a proximal section. The balloon member is attached at its proximal and distal sections to the tapered distal tip of the inner member. The proximal section of the balloon member extends internally of the radially expandable scaffold member at the distal end of the outer sheath. The balloon member can assume intermittently an inflated configuration and deflated configuration. When the balloon member is controlled to assume the inflated configuration by actuating a balloon inflation system, the proximal section of the balloon member expands and causes the radially expandable scaffold member to assume its opened configuration.

The subject method further includes the following steps of the reverse CART surgery:
Step B: performing an antegrade dissection of a blood vessel of interest to form a subintimal space next to an occlusion in the blood vessel of interest;
Step C: inserting an antegrade guide wire into the subintimal space from an antegrade end of the blood vessel of interest;
Step D: inserting a retrograde guidewire into the subintimal space from a retrograde end of the blood vessel of interest,
Step E: extending the modified guide wire catheter extension system over the antegrade guidewire in the subintimal space,
Step F: actuating an inflation system to inflate the balloon member, thus plastically deforming the radially expandable scaffold member to transform into its opened configuration;
Step G: deflating the balloon member;
Step H: retracting the inner member from the outer member,
Step I: inserting a distal end of the retrograde guidewire in the expanded distal opening of the radially expandable scaffold member at the distal end of the outer member, and
Step J: entering a retrograde catheter/microcatheter into the expanded distal opening of the radially expandable scaffold member of the outer member and advancing the retrograde catheter inside and along the outer member beyond the proximal end of the outer sheath.

The reverse CART procedure further includes the steps of: in the step J, advancing a retrograde microcatheter into the expanded distal opening of the radially expandable scaffold member, removing the retrograde guide wire from the outer sheath of the outer member, and advancing the retrograde catheter into the retrograde micro-catheter and out of the outer sheath of the outer member.

Subsequent to the step J, angioplasty and subsequent coronary stenting are performed for coronary revascularization to reconstruct an occlusion in the blood vessel of interest from a distal true lumen to a proximal true lumen at both sides of the subintimal space.

In still a further aspect, the present invention constitutes an enhanced method for removal of a thrombus from a blood vessel of interest by using a modified guide catheter extension system. The subject method includes the Step A for assembling a modified intravascular guide catheter extension system configured with:
an outer member (also referred to herein as a thrombus removing catheter) formed by a flexible substantially cylindrically contoured elongated outer sheath defining an outer sheath lumen having a proximal end and a distal end,
a radially expandable scaffold member positioned at an outer tip at the distal end of the outer sheath. The radially expendable scaffold member may be configured with a plastically deformable elongated member (wire) which is shaped into a zig-zag configuration to form a plurality of wing members. Each wing member extends longitudinally of the outer sheath, and the number of the wing members are disposed circumferentially along the walls of the outer sheath around a longitudinal axis of the outer sheath.

The radially expandable scaffold member can intermittently assume a closed configuration and an opened configuration. In the closed configuration, the wing members of the radially expandable scaffold member are arranged in a cylindrical tubular formation having a proximal opening and a distal opening with approximately equal diameters. In the opened configuration, the scaffold member is plastically deformed to radially expand the distal ends of the wing members from one another to enlarge the distal opening formed by the distal ends of the wing members of the radially expandable scaffold member. The shape memory elongated member (wire) of the scaffold member maintains the opened configuration as required by the thrombus removal procedure.
an inner member (also referred to herein as a balloon catheter) which has an elongated body defining an internal channel extending along its longitudinal axis. The inner member extends internally along the outer sheath lumen of the outer sheath of the outer member in a controllable relationship with the outer sheath. The inner member has a tapered distal tip configured with a tapered delivery micro-catheter having an elongated body of a predetermined length. The tapered delivery micro-catheter can be displaced along the guide wire beyond the distal end of the outer sheath.
a balloon member having a distal section and a proximal section. The balloon member is attached at its proximal and distal sections to the tapered distal tip of the inner member. The proximal section of the balloon member extends internally of the radially expandable scaffold member at the distal end of the outer sheath. The balloon member can assume intermittently an inflated configuration and a deflated configuration. When the balloon member is controlled to assume the inflated configuration by actuating a balloon inflation system, the proximal section of the balloon member expands and causes the radially expandable scaffold member to assume its opened configuration.
a connection mechanism controllable by a surgeon to engage or disengage the inner and outer members either for the common motion of the inner and outer members (in the engaged configuration of the modified guide catheter extension system) or for the separate displacement of the inner and outer members (in the disengaged configuration of the modified guide catheter extension system).

The subject method further includes the following steps of the enhanced thrombus removal procedure:
Step B: inserting a guide wire into the blood vessel of interest towards the location of the thrombus in the blood vessel of interest;
Step C: actuating the connection mechanism to obtain the engaged configuration of the of the modified guide catheter extension system;
Step D: displacing the modified guide wire catheter extension system in the engaged configuration within the blood vessel of interest with the inner member sliding over the guidewire towards the thrombus in the blood vessel of interest;
Step E: upon arriving with the inner catheter’s micro-catheter to the desired location in proximity to the thrombus, actuating an inflation system to inflate the balloon member, resulting in a plastic deformation of the radially expandable scaffold member and transformation into its opened configuration.

In Step A, the balloon member may be formed from a balloon material loaded with tungsten, and/or barium, gold, or other radiopaque material which is prefabricated with a plurality of micro pores, and in Step E, the balloon member may be inflated with a medicinal fluid (for example, thrombolytic agent), and a pressure may be created inside the balloon member sufficient to expel the medicinal fluid from the balloon member through the plurality of micro pores to the thrombus to soften (breaking the thrombus) before removal.

The subject method further continues by Step F for deflating the balloon member and de-actuating the connection mechanism to convert the modified guide wire catheter extension system into the disengaged configuration;
Step G: retracting the inner member from the outer member; and
Step H: coupling to the proximal end of the outer member and actuating the vacuum system/syringe to aspirate the thrombus (which is softened or broken up by the delivered thrombolytic agent) through the expanded distal opening of the radially expandable scaffold member at the distal end of the outer member and internally through the outer member lumen to remove the thrombus at the proximal end of the outer member. To provide a rotation ability to the subject system between right and left pulmonary arteries, in Step A, the outer sheath of the outer member or an inner member may be pre-shaped with a curved portion at the distal. The curved portion preferably deviates from a longitudinal axis of the outer sheath or inner member at the proximal end at an angle ranging between 30° and 90°.

These and other novel features and advantages of the present invention will be apparent in view of further description in conjunction with the accompanying Patent Drawings.

### BRIEF DESCRIPTION OF THE DRAWINGS

FIGS. 1A-1F represent the typical coronary reverse CART technique performed for percutaneous intervention in peripheral and coronary arteries in the cases of open chronic total occlusion (CTO);

FIGS. 2A-2C are representative of the distal portion of the subject guide catheter extension system with FIG. 2A showing the inner catheter, FIG. 2B showing the outer catheter, and FIG. 2C showing the assembled inner and outer catheters of FIGS. 2A and 2B, respectively;

FIGS. 3A-3C are representative of the mid-shaft and proximal portions of the subject guide catheter extension system, with FIG. 3A showing the inner catheter, FIG. 3B showing the outer catheter, and FIG. 3C showing the assembled inner and outer catheters of FIGS. 3A and 3B, respectively;

FIG. 4 depicts the interaction between the outer catheter and inner catheter with the radially expandable scaffold member in its closed configuration and the balloon member in its deflated configuration;

FIG. 5 depicts the inter-relationship between the inner and outer catheters of the subject guide catheter extension system with the balloon member inflated and the radially expandable scaffold member in its opened configuration;

FIG. 6 is a representation of the distal end of the outer catheter with the inner catheter removed and with the distal end of the outer catheter forming a funnel-like distal opening;

FIGS. 7A-7I represent the sequence of steps of the subject reverse CART surgery using the subject modified guide catheter extension system;

FIGS. 8A-8E represent the sequence of steps of the subject enhanced thrombus removal procedure using the modified guide catheter extension system of the present invention;

FIG. 9 is a schematic representation of an alternative embodiment of the subject guide catheter extension system showing the outer catheter configured with a pre-shaped curved portion at the distal end which may be beneficial for rotational ability of the catheter between the right and left pulmonary arteries;

FIGS. 10A and 10B show schematically an external view (FIG. 10A) and a longitudinal cross-sectional view (FIG. 10B) of another alternative embodiment of the subject guide catheter extension system with a balloon member fabricated from a radiopaque material loaded balloon member material with micro pores for delivery of a thrombolytic agent to a thrombus in a pulmonary or coronary artery; and

FIG. 11 shows schematically the process of the medicinal agent exit from the balloon through the micro pores fabricated in the balloon member of FIGS. 10A-10B.

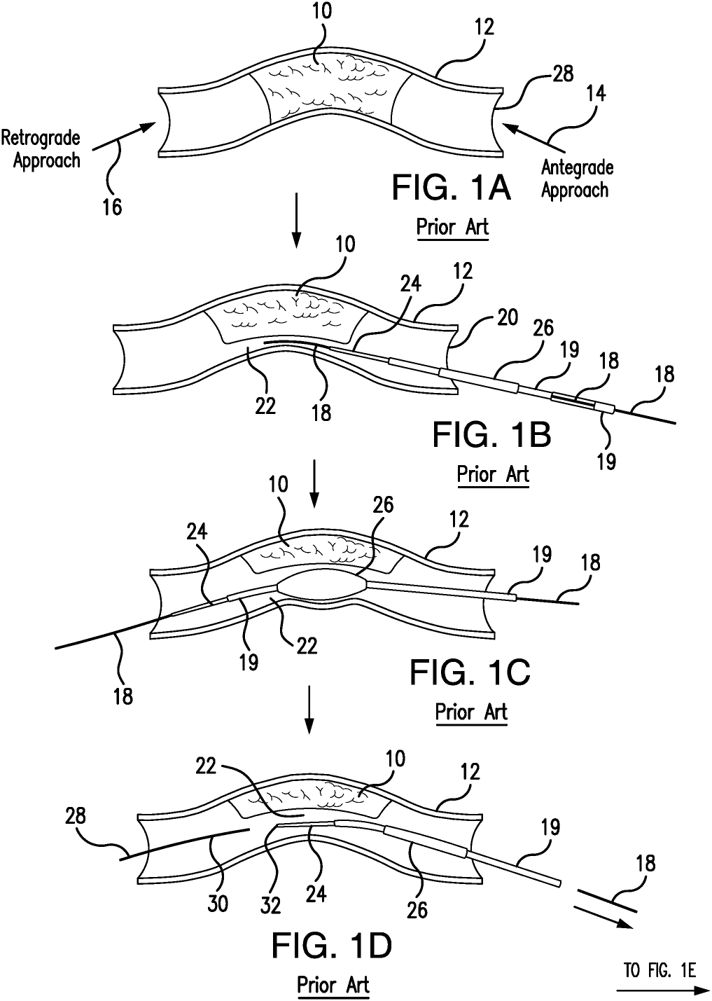

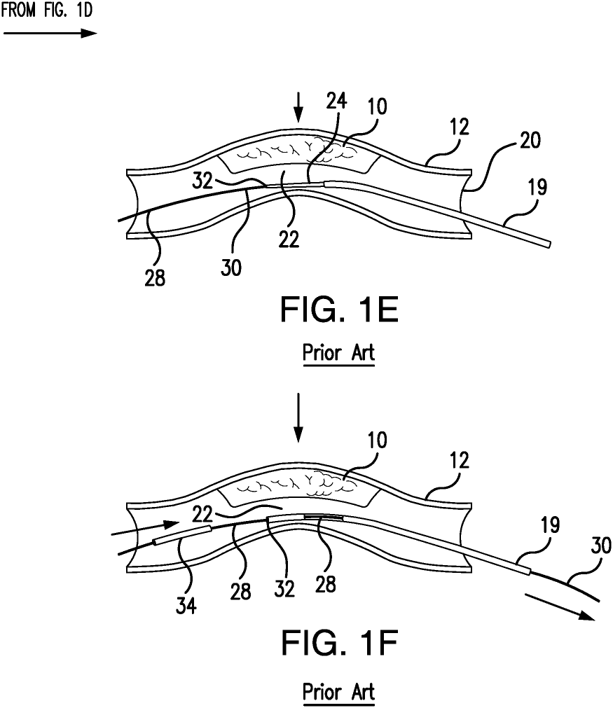

### DETAILED DESCRIPTION OF THE PREFERRED EMBODIMENT(S

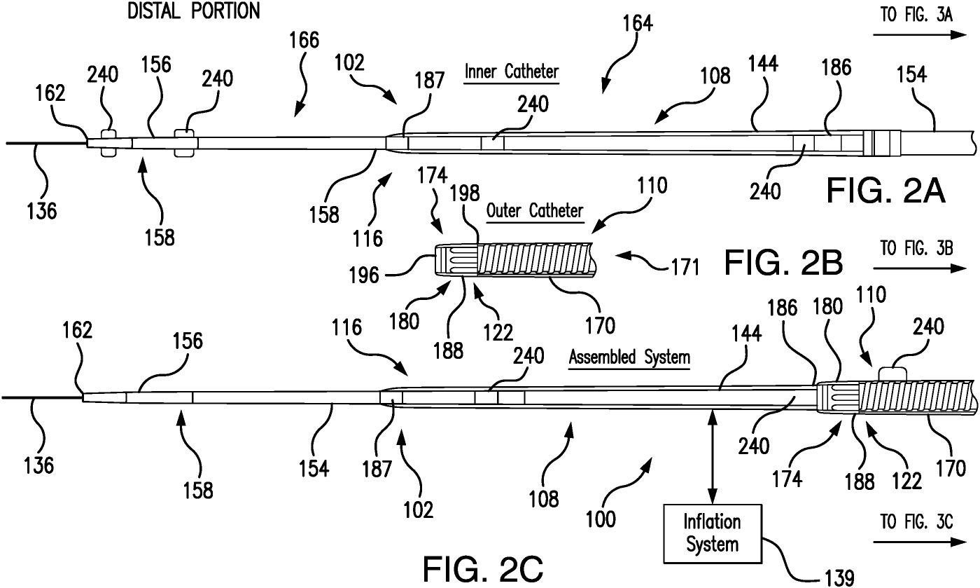

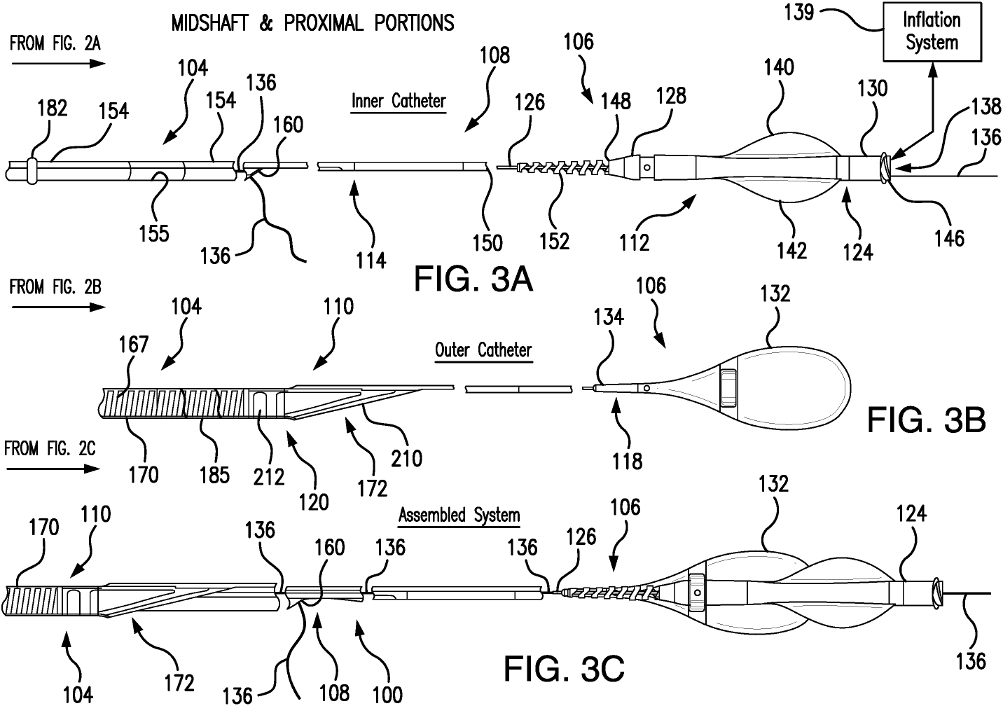

FIGS. 2A-2C and 3A-3C depict a subject guide catheter extension system 100 having a distal portion 102, a mid-shaft portion 104, and a proximal portion 106. The guide catheter extension system 100 includes an inner catheter 108 shown in FIGS. 2A and 3A, and an outer catheter 110 detailed in FIGS. 2B and 3B. The assembly of the inner catheter 108 and the outer catheter 110 is depicted in FIGS. 2C and 3C.

The subject guide catheter extension system 100 has been designed with the goal to enhance intravascular surgeries, including but not limited to reverse CART technique, thrombus removal procedure, or other intravascular procedures. The present modified guide catheter extension system 100 constitutes a modified design of the Crossliner™ guide catheter extension system described in U.S. Pat. Applications Serial No. 15/899,603 filed 20 Feb. 2018; Serial No. 16/132,878 filed 17 Sep. 2018; and Serial No. 16/793,120, filed 18 Feb. 2020, currently pending, which are incorporated herein by reference in their entirety.

As a specific example of the present modified guide catheter extension system 100 application for an intravascular procedure, such will be presented in conjunction with the reverse CART procedure and the thrombus removal procedure. However, the usage of the subject system 100 in other intravascular procedures is also contemplated.

As shown in FIGS. 2A and 3A, as well as 2C and 3C, the subject inner catheter 108 includes a proximal section 112, a middle section 114, and a distal section 116. As shown in FIGS. 2B and 3B, as well as 2C and 3C, the outer catheter 110 has a proximal end 118, middle shaft 120, and the distal end 122.

Referring to FIGS. 3A and 3C, at the proximal section 112, the inner catheter 108 is represented by a proximal handle 124 of the inner catheter 108. A proximal pusher 126 is connected between the distal end 128 of the proximal handle 124 and the tubular body 130 of the inner catheter 108.

During the intravascular intervention, the proximal handle 124 of the inner catheter 106 is manipulated by a surgeon (operator) who performs the coronary procedure to position the guide catheter extension system 100 at a desired location within the vessel of interest, as well as to advance or redirect the inner catheter 108 as required by the coronary intervention procedure.

Referring to FIGS. 3B and 3C, a proximal handle 132 is positioned at the proximal end 118 of the outer catheter 110. The proximal handle 132 is connected by an outer member pusher 134 to the middle shaft 120 of the outer catheter 110. During the procedure, the proximal handle 132 of the outer catheter 110 along with the proximal handle 124 of the inner catheter 108 are manipulated by a surgeon performing the coronary intervention procedure to slide the guide catheter extension system 100 inside the vessel of interest to position the distal section 116 of the inner catheter 108 into the subintimal space as required by the reverse CART procedure, as well as to advance or retract the inner catheter 108 and the outer catheter 110 relative to one another, or to slide the guide catheter extension system 100 along an antegrade guide wire 136 as required by the coronary intervention procedure.

As shown in FIGS. 3A and 3C, the proximal handle 124 of the inner catheter 108 is formed with a central channel 138 (which constitutes a portion of a inflation passage) and two tabs 140 and 142 (which provide convenience for a surgeon while manipulating the inner catheter 108). By providing the proximal handle 124 of the inner catheter with the central channel 138, the proximal handle 124 also performs the function of an inflation hub. The central channel 138 (also referred to herein as the internal inflation channel) serves as a passage for inflation of air (or other fluids) between a balloon inflation system 139 and a balloon member 144 integrated with the inner catheter 108 for the controlled inflation/deflation of the balloon member 144 as prescribed by the cardiac procedure, as will be detailed in future paragraphs.

The central channel 138 may have a cone-shaped configuration and is connected by its proximal opening 146 to the balloon inflation system 139. The central channel 138 is also configured with a distal opening 148 which is coupled to an inflation lumen hypo-tube 150 which extends through the length of the proximal section 112 of the inner catheter 108 and along the middle portion 104 of the guide catheter extension system 100. The central channel 138terminates at the distal section 116 of the inner catheter 108, particularly in fluid communication with the balloon member 144.

The inner catheter pusher 126 has serrated flexible member 152 which supports the proximal end of the inflation lumen hypo-tube 150 and provides a flexible bending of the structure when manipulated by a surgeon.

The inner catheter 108 is provided with the inner catheter shaft 154 which extends between the inflation lumen hypo-tube 150 through the middle portion 104 of the guide catheter extension system 100 and through the distal section 116 of the inner catheter 108 and terminates with a microcatheter 156 and the distal end 158 of the inner catheter shaft 154.

The inner catheter shaft 154 is configured with a rapid exchange (RX) guidewire (GW) port 160 in proximity to the connection of the inner catheter shaft 154 and the inflation lumen hypo tube 150. A guidewire lumen 155 begins with its proximal end at the RX port 160 and extends between the RX port 160 inside the inner catheter shaft 154 through the entire length of the distal section 116 of the inner catheter 108. The guidewire lumen 155 forms an internal channel with the proximal end corresponding to the RX port 160 and a distal end corresponding to the outermost distal end 162 of the distal section 116 of the inner catheter 108. The distal end of the guidewire lumen constitutes a gradually tapered portion 164 which may be in the form of the microcatheter 156.

The inner catheter 108 is configured with a tapered distal portion 166 at the distal section 116. The tapered distal portion 166 is equipped with the balloon member 144 which is secured onto the tapered distal portion 166. The balloon member 144 has a proximal section 186 and a distal section 187 and is secured to the inner catheter’s tapered distal portion (also referred to herein as the tip) 166 at the proximal and distal sections, 186 and 187, respectively.

During the displacement of the guide catheter extension system 100 within the blood vessel of interest, the balloon member 144 is maintained in deflated (folded) configuration. Upon arriving at the target site within the blood vessel of interest, specifically, when the guide catheter system 100 is delivered to the subintimal space, the balloon member 144 is inflated through the inflation hub/proximal handle 124, central channel 138, and the inflation lumen hypo-tube 150 by actuating the inflation system 139.

Referring now to FIGS. 2B and 3B, as well as FIGS. 2C and 3C, the guide catheter extension system 100 includes the outer catheter 110 which includes an outer sheath 170 having an outer sheath lumen (or channel) 171 with a proximal end 172 connected to the outer catheter pusher 134. The outer sheath 170 of the outer catheter 110 is fabricated with a flexible cylindrically shaped tubular body 167 extending substantially the length of the middle portion 104 of the subject system 100. The outer sheath lumen 171 of the outer sheath 170 also has a distal end 174 configured with a plastically deformable member 180 which can elastically deform as detailed in the following paragraphs.

A coupler mechanism 182 is formed between the outer surface 184 of the inner catheter shaft 154 and the inner surface185 of the sheath 170 of the outer catheter 110. The coupler mechanism 182 is contemplated in several embodiments detailed in the description of the Crossliner™ guide catheter extension system presented in U.S. Pat. Applications Serial No. 15/899,603 filed 20 Feb. 2018; Serial No. 16/132,878 filed 17 Sep. 2018; and Serial No. 16/793,120, filed 18 Feb. 2020, currently pending, which are incorporated herein by reference in their entirety.

During the procedure, the inner catheter 108 is inserted within the outer catheter 110 with the inner catheter shaft 154 inserted within the sheath 170, as shown in FIGS. 2C and 3C. The balloon member 144 has its proximal section 186 disposed internally within the distal end 174 of the sheath 170 of the outer catheter 110. The plastically deformable member 180 at the distal end 174 of the sheath 170 of the outer catheter 110 snugly embraces the proximal section 186 of the balloon 144.

The plastically deformable member 180 at a distal end 174 of the sheath 170 of the outer catheter 110 is formed by an elastic material forming a distal sheath 188 having a tubularly shaped portion 190 extending over the distal end 174 of the sheath 170 and the proximal end 186 of the balloon member 144. The distal sheath 188 of the plastically deformable member 180 also has a tapered portion 192 which extends from the tubularly shaped portion 190 of the distal sheath 188 towards the proximal end 186 of the balloon member 144 to snugly embrace the latter.

The distal sheath 188 is integrated with an elongated member, also referred to herein as a shape memory wire, 200 which is configured in a zig-zag configuration to form a radially expandable scaffold member 194 which is embedded into the tubularly shaped portion 190 of the plastic distal sheath 188. The wire 200 may be fabricated from a plastically deformable material. As an example, the wire 200 in the scaffold member 194 may be fabricated from stainless steel, cobalt chromium, Nitinol, or other alloys and plastics, and their combinations, which demonstrate plastic deformation properties. The wire 200 in the scaffold member 194 is shaped to form numerous wing-like members 201, each having a distal end 203 and a proximal end 205. The wing-like members 201 are oriented in a longitudinal direction of the scaffold member 184 and are disposed circumferentially along the longitudinal axis 207 of the scaffold member 194.

The radially expandable scaffold member 194 is configured with a distal end 196 and a proximal end 198. The proximal ends 205 of the wing-like members 201 form a proximal opening 209 at the proximal end 198, and the distal ends 203 of the wing-like members 201 form a distal opening 211 at the distal end 196 of the scaffold member 194. The scaffold member 194 can intermittently assume a closed configuration and an opened configuration.

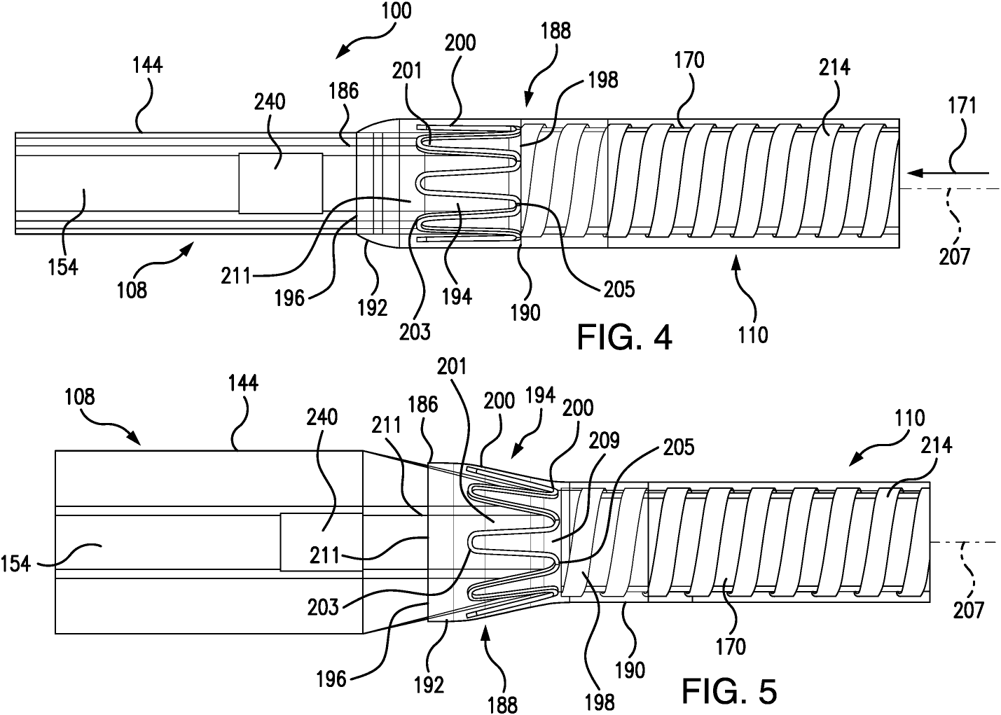

Due to the elasticity of the wire 200 forming the radially expandable scaffold member 194, the scaffold 194 expands and contracts when needed. As shown in FIGS. 2C and 4, in original (closed) configuration, the diameters of the distal opening 211 at the distal end 196 and the proximal opening 209 at the proximal end 198 of the scaffold member 194 are approximately equal to one another.

Subsequent to the inflation of the balloon member 144, as required by the subject reverse CART procedure, the proximal section 186 of the balloon member 144 expands, as shown in FIG. 5, to result in elastic expansion of the tubularly shaped portion 190 and the tapered portion 192 of the plastic distal sheath 188 with simultaneous deformation of the scaffold member 194 where the distal ends 203 of the wing-like members 201 space apart one from another, thus expanding the diameter of the distal opening 211 and enlarging the distal end 196 of the expandable scaffold member 194. When the balloon member 144 is inflated, as best shown in FIG. 5, the wire 200 of the scaffold member 194 plastically deforms so that the scaffold member 194 assumes an open configuration, shown in FGIS. 5 and 6, with the diameter of the distal opening 211 larger than its original diameter.

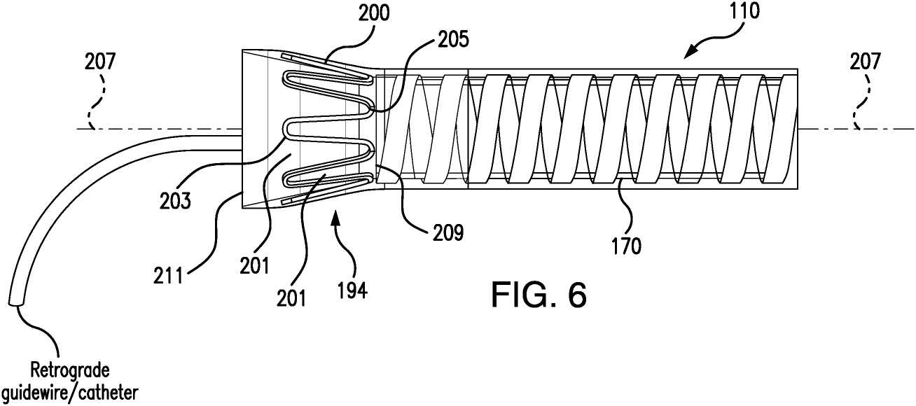

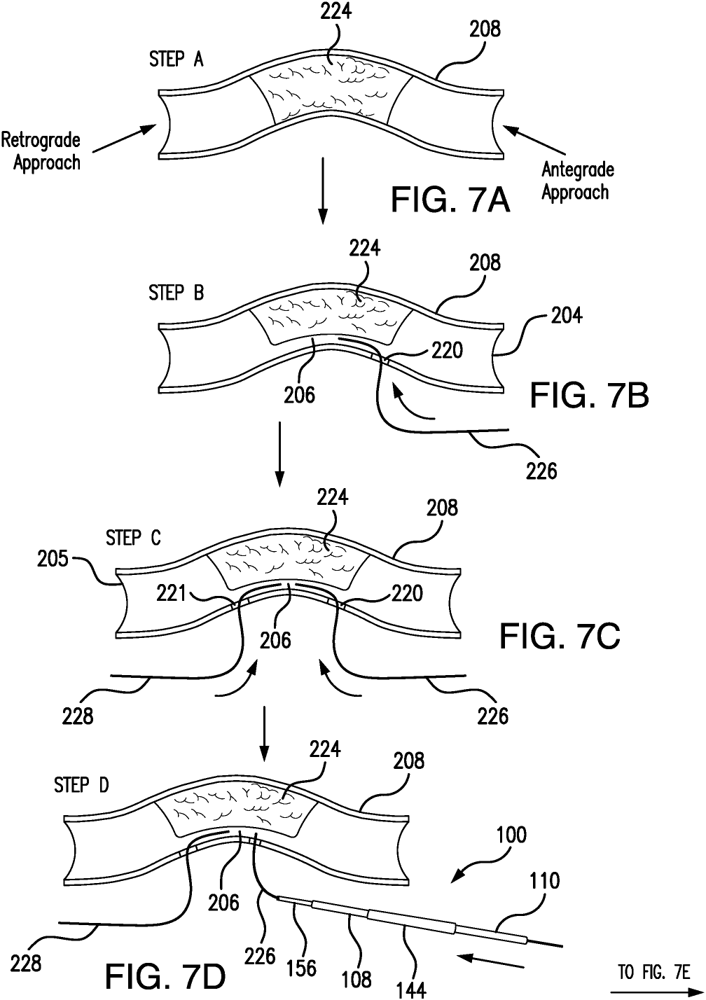

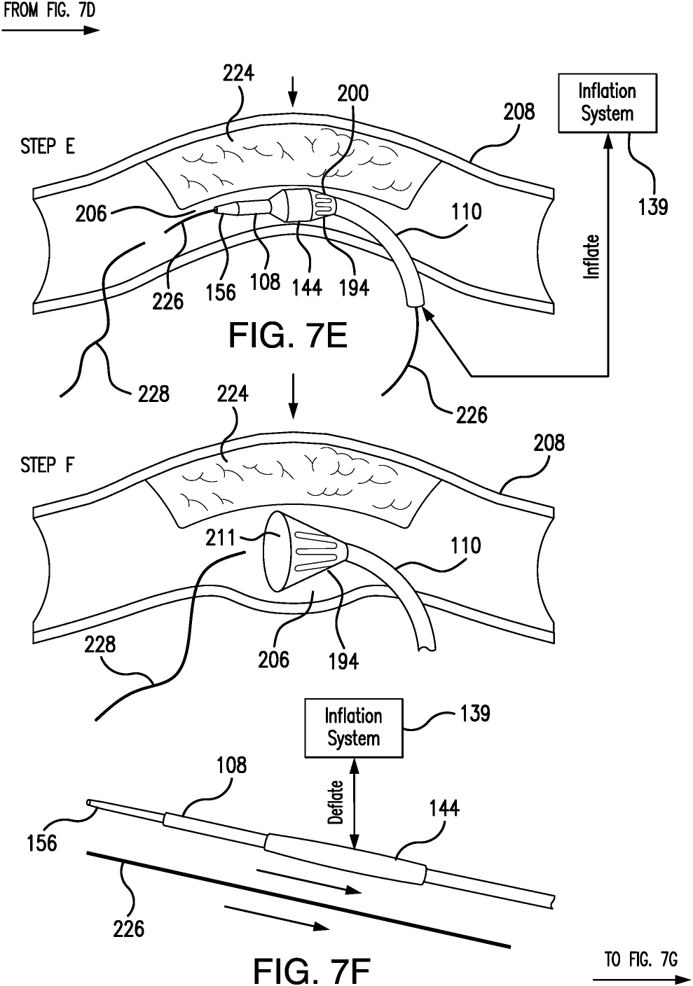

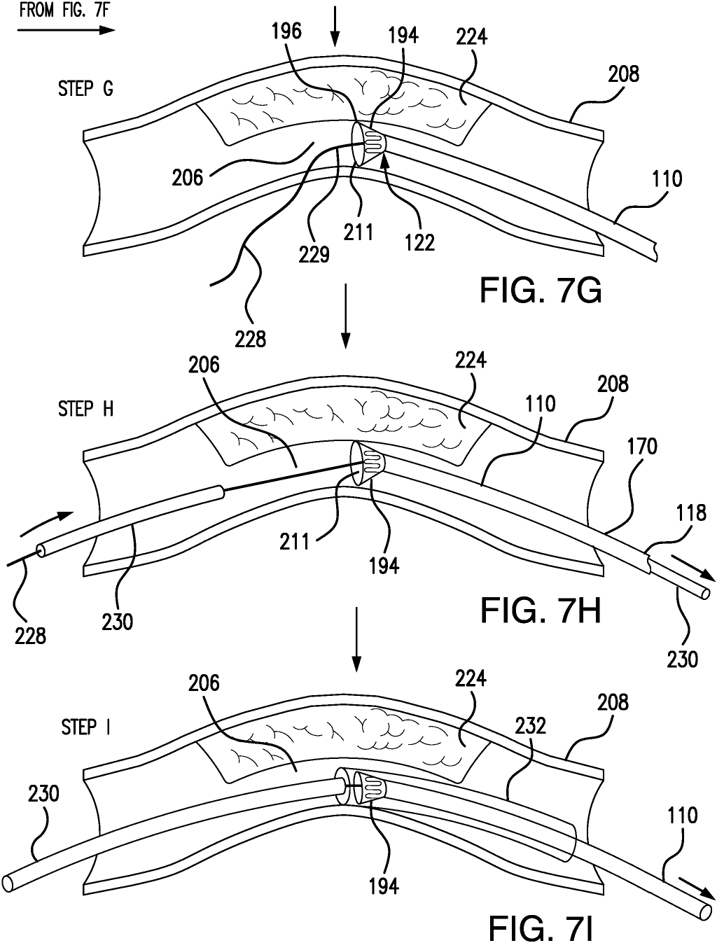

In the opened configuration of the scaffold member 194 shown in FIGS. 5 and 6, the balloon member 144 is subsequently deflated, and the inner catheter 108 is removed (retracted) from the sheath 170 of the outer catheter 110. Subsequent to removal of the inner catheter 108 from the outer catheter 110, the scaffold member 194 remains in the opened configuration, as shown in FIG. 6, due to the shape memory property of the wire 200. In the opened configuration, the enlarged distal opening 211 provides an enhanced “catching” capability for the retrograde wire (or catheter) to be inserted into the distal opening 211 of the scaffold member 194 in the subintimal space 206 shared by the antegrade and retrograde systems in the target vessel 208, as shown in FIG. 7 F. When, as required by the surgical procedure, the outer sheath 170 is to be removed from the blood vessel 208, as shown in FIG. 7I, the wing members 201 are plastically compressed by the walls of the guide wire 232, and the outer sheath 170 along with the scaffold member 194 easily pass along the internal lumen of guide catheter 232.

The subject system 100 is built with an interconnection mechanism 210 at the middle portion 104. The interconnection mechanism 210 may include a proximal coupler 212 formed at the proximal end 172 of the sheath 170 of the outer catheter 110, and a cooperating (coupler) mechanism 182 formed at the outer surface of the inner catheter 108 as depicted in FIG. 3A.

The subject system may operate in an inner/outer catheter’s engagement mode and in an inner/outer catheter’s disengagement mode, which is accomplished by controlling the interconnection mechanism 210. The subject interconnection mechanism 210 is configured to engage/disengage the inner and outer catheters 108, 110, as required by the cardiac procedure, as well as to prevent an unwanted displacement of the inner catheter 108 inside the outer delivery sheath 170 of the outer catheter 110. The engagement mode of operation allows the enhanced “pushability” of the system as a whole (with the outer catheter 110 connected and locked to the inner catheter 108, as shown in FIGS. 2C and 3C), even with the connected pushing/pulling element (pusher) of the outer catheter 174 of the outer catheter 110 configured as a low profile and flexible element (as flexible or more flexible than the outer tubular sheath 170 of the outer catheter 110).

The connection unit 210 may operate based on the interference between the proximal coupler 212 configured at the proximal end 172 of the sheath 170 and the cooperating mechanism 182 configured at the outer surface of the inner catheter 104 when the inner surface of the sheath 170 (at its proximal end 172) engages the outer surface of the cooperating mechanism 182 of the inner catheter 108.

A number of interconnection mechanisms 210 are envisioned to be applicable to the subject guide catheter extension system 100 for controllable engagement/disengagement between the inner catheter 108 and the outer catheter 110, as well as to prevent a forward motion of the inner catheter 108 relative to the outer delivery sheath 170 beyond a predetermined position. The examples of the interconnection mechanism may be found in the Crossliner™ described in U.S. Pat. Applications Serial No. 15/899,603 filed 20 Feb. 2018; Serial No. 16/132,878 filed 17 Sep. 2018; and Serial No. 16/793,120, filed 18 Feb. 2020, currently pending, which are incorporated herein by reference in their entirety.

Various forms of reinforcing the sheath 170 of the outer catheter 110 may be envisioned in the present structure. For example, the sheath 170 may be reinforced by manufacturing with braid reinforcement structure which may create a somewhat flexible tubing. Alternatively, the reinforcement structure may be configured with various metallic patterns or wires. The metal braid may be embedded into the reinforced walls of the sheath 170 to add increased flexibility thereto required for a retraction of the inner catheter 108 relative to the outer delivery sheath 170 during the procedure.

A flat wire helical coil made for example from a shape memory alloy, such as Nitinol, with a wire thickness of approximately 1 mil - 3 mil which may be embedded into the braid. This coil may be formed with a very thin coating of plastic placed onto its inner and outer surfaces, which facilitates the reduction of the wall thickness of the inflation lumen distal shaft to less than 7 mils and preferably to approximately 5 mils.

The reinforcing of the tubular members (outer sheath 170) in the subject system may be attained by the catheter shaft coil reinforcement 214 in the form of a flat wire having a helical coil, or forming the tubular members from the flat wire helical coil, as shown in FIGS. 3A and 3B and 2C and 3C. Such reinforcement also can be applied to the microcatheter 156 at the distal end 158 of the inner catheter shaft 154. In the outer delivery sheath 170 and/or the microcatheter 156, such flat wire helical coil may be embedded in predetermined positions along the length of wall thereof, for example, at the proximal end or distal ends.

Alternatively, the entire length of the outer delivery sheath 170 and/or microcatheter 156 may be formed with a flat wire helical coil. The pitch between the coils may be adjusted to provide either the flexibility gradient along the length of the tubular member increasing towards the distal end thereof to facilitate a traumatic operation, or to provide rigidity to the microcatheter 156 during dissection through the occlusion lesion in the blood vessel 208.

For visualization of the location of the present guide catheter extension system 100 in the blood vessel 208 a number of radio-opaque markers 240 may be attached to various parts/elements of the guide catheter extension system 100. For example, the radio-opaques markers 240 may be provided at the distal end 174 of the outer sheath 170, a distal end 162 of the micro-catheter 156, the tapered distal section 116 of the inner catheter 108 in proximity to the proximal section 186 and distal section 187 of the balloon member 144, and other locations which may be beneficial for visualization of the cardiac procedure.

Referring now to FIG. 7A, depicting a Step A of the subject reverse CART procedure, the operation is performed on a blood vessel (artery) 208 which is blocked with a blockage 224 which completely occludes the blood vessel 208.

Subsequently, in Step B shown in FIG. 7B, an antegrade dissection 220 is made in proximity to the blockage 224 by a surgeon in the antegrade approach in the blood vessel 208 having a chronic total occlusion lesion 224 to form an opening underneath the blockage 224 (which is schematically shown as a subintimal space 206), and an antegrade guide wire 226 is introduced into the subintimal space 206 in the blood vessel 208 through the antegrade dissection 220 (in the direction from a proximal end 204 of the blood vessel 208).

As depicted in FIG. 7C, in the following Step C, a retrograde dissection 221 of the blood vessel 208 is made in the retrograde direction, and a retrograde guidewire 228 is introduced into the space 206 through the retrograde dissection 221(in the direction from a distal end 205 of the blood vessel 208 of interest).

Subsequently, as shown in FIG. 7D, in Step D, the subject guide wire catheter extension system 100 (with the balloon member 144 in its deflated state, as depicted in FIG. 4) is extended over the antegrade guidewire 226.

In Step E, shown in FIG. 7E, the inflation system 139 is actuated to inflate the balloon member 144. The inflation of the balloon member 144 results in the plastic deformation of the radially expandable scaffold member 194 to transform into its opened configuration, as shown in FIG. 5. Subsequently, the antegrade guidewire 226 is removed, the balloon member 144 is deflated, and in Step F, as shown in FIG. 7F, the inner catheter 108 is retracted through the outer member 110 from the vessel 208. The scaffold member 194 remains in its opened configuration with the expanded distal opening 211, as shown in FIG. 6 (which may be due to the plasticity of the wire 200 or due to the shape memory of the wire 200) even after the balloon member 144 is transformed into the deflated configuration, and the inner catheter 108 is removed from the system 100 in the blood vessel 208. Although not shown to a precise dimensional scale, it is contemplated that when the scaffold member 194 is in its opened configuration, it is expanded approximately to ⅔ of the dilated dissected blood vessel (artery) diameter.

In Step G, as shown in FIG. 7G, a distal end 229 of the retrograde guidewire 228 is inserted in the enlarged distal opening 211 at the distal end 196 of the scaffold member 194 positioned at the distal end 122 of the outer member 110.

In the subsequent Step, H, as depicted in FIG. 7H, a retrograde catheter/microcatheter 230 slides over the retrograde guidewire 228 into the expanded distal opening 211 of the scaffold member 194 of the outer member 110, and the retrograde catheter 230 is advanced inside and along the outer member 110 beyond the proximal end 118 of the outer sheath 170.

In the subsequent Step I, shown in FIG. 7I, the outer catheter 110 is retracted from the vessel 208 inside the guide catheter 232. Due to the elastic property of the wire 200, the wing members 201, being compressed by the walls of the guide catheter 232, result in slight reduction of the distal end 196 of the scaffold member 194, sufficient for easy removal of the outer catheter 110 from the guide catheter 232.

Subsequent to the Step I, the angioplasty followed by the coronary stenting are performed for coronary revascularization to reconstruct an occlusion in the blood vessel of interest from a distal true lumen to a proximal true lumen at both sides of the subintimal space.

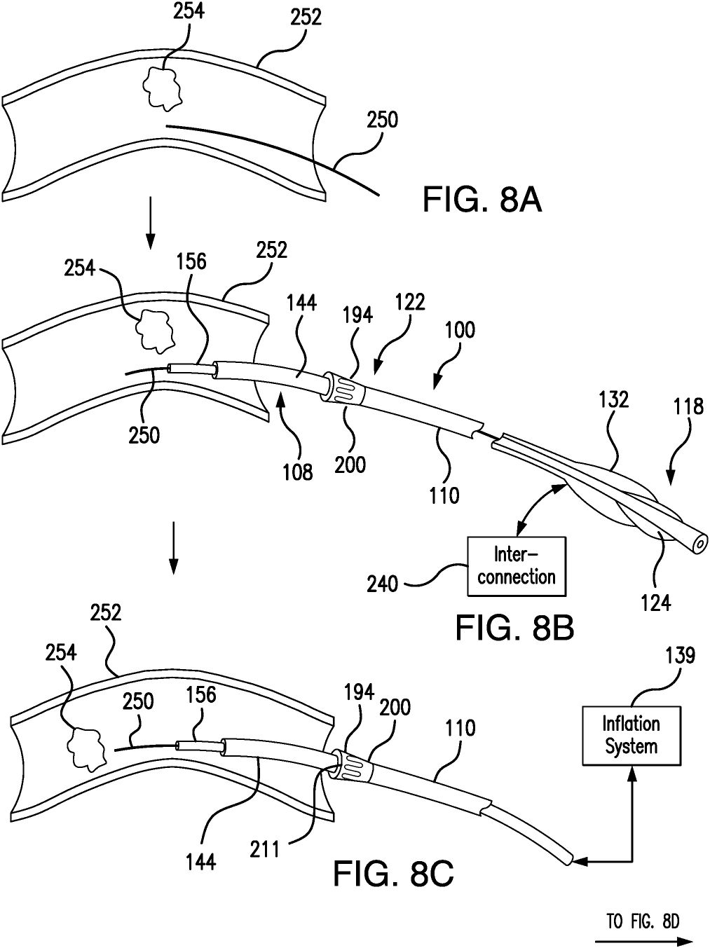

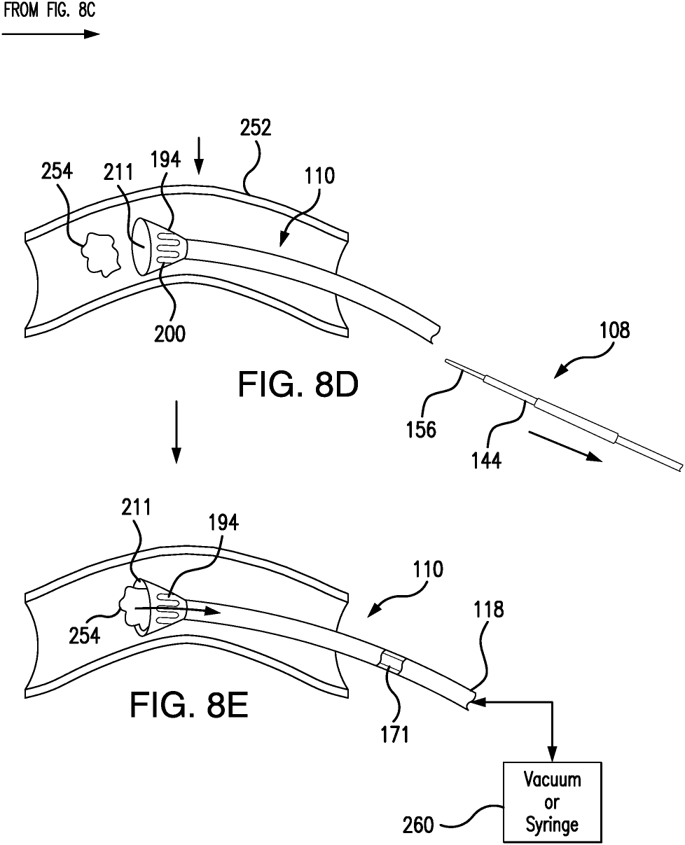

The present modified guide catheter extension system 100 is also applicable to a thrombus removal procedure, as presented in FIGS. 8A-8E. By using the subject modified guide catheter extension system 100, an enhanced surgery for removal of a thrombus from a blood vessel of interest may result due to the rapid catching of the thrombus into the expanded distal opening.

Referring to FIG. 8A, the subject thrombus removal method includes the step of inserting a guide wire 250 into the blood vessel 252 towards the location of the thrombus 254. In the subsequent step shown in FIG. 8B, the modified guide catheter extension system 100 in its engaged configuration (by actuating the interconnection mechanism 240) and with the balloon member 144 in the deflated configuration advances inside the blood vessel 252 with the micro-catheter 156 of the inner catheter 108 sliding over the guide wire 250. The interconnection mechanism 240 may be in any configuration contemplated and described herein. A surgeon may manipulate the proximal handle 124 of the inner catheter 108 and/or the proximal handle 132 of the outer catheter 110 to displace the modified guide catheter extension system 100 inside the blood vessel 252.

As shown in FIG. 8C, upon arriving with the inner catheter’s micro-catheter 156 to the desired location in proximity to the thrombus 254, the inflation system 139 is actuated to inflate the balloon member 144, resulting in a plastic deformation of the wire 200 in the radially expandable scaffold member 194 and transformation into its opened configuration with the expanded distal opening 211 at the distal end 122 of the outer catheter 110.

Subsequently, the balloon member 144 is deflated by the inflation system, and the interconnection mechanism is de-actuated to convert the modified guide wire catheter extension system 100 into the disengaged configuration, and as shown in FIG. 8D, the inner member 108 is retracted from the outer member 110.

In the subsequent step shown in FIG. 8E, a vacuum system (or a syringe) 260 is operatively coupled to the proximal end 118 of the outer catheter 110. The vacuum system/syringe 260 is actuated to aspirate the thrombus 254 through the expanded distal opening 211 of the radially expandable scaffold member 194 at the distal end 122 of the outer member 110 and internally through the outer member lumen 171 to remove the thrombus 254 at the proximal end 118 of the outer member 110.

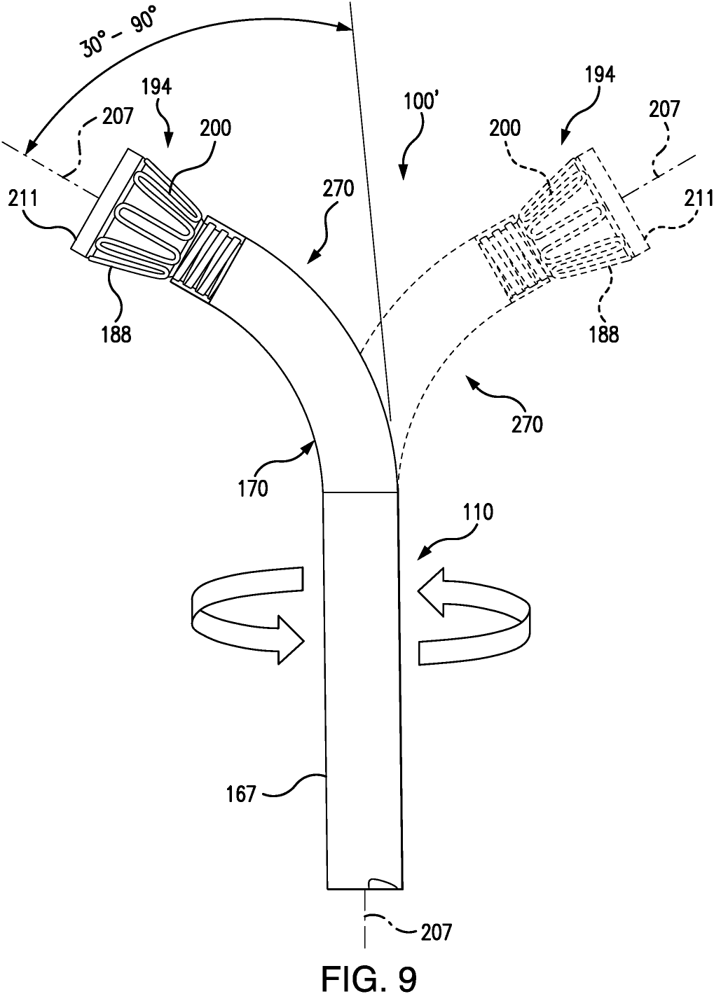

It is sometimes difficult to rotate a catheter from one pulmonary artery into the other main pulmonary artery. FIG. 9 is representative of an additional implementation of the subject guide catheter extension system 100' designed to facilitate the rotation of the guide catheter extension system 100' from a right pulmonary artery to the left or vice versa during surgical procedure, that is especially beneficial for the thrombectomy procedure. As shown in FIG. 9, the subject guide catheter extension system 100' may be configured with the outer catheter 110 having a pre-shaped curved portion 270 at its distal end 122. The outer catheter’s polymer jacket (outer sheath) 170 (for example, fabricated from Pebax) may be formed into the curved shape 270 using a heated die/forming mold. The angle of the curvature, i.e., the angle between the axis 207 and the longitudinal direction of the tubular body 167 of the outer catheter 110 may be an acute or smooth angle from 30° to 90°. Alternatively, instead of the curved outer catheter, the inner catheter 108 may be configured with a pre-shaped or deflectable curve at its distal end.

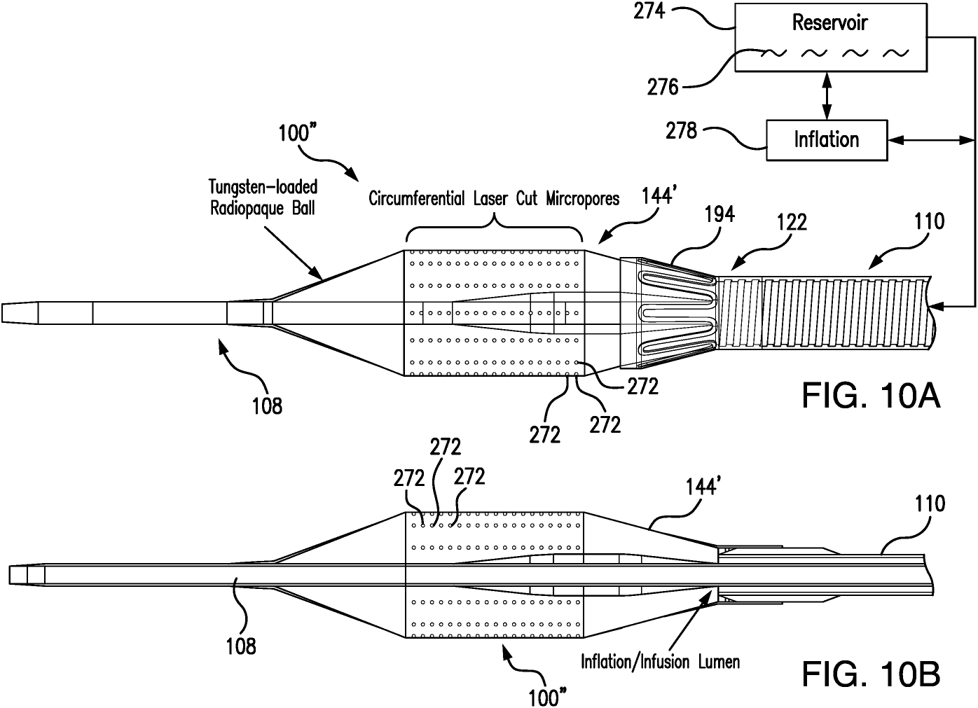

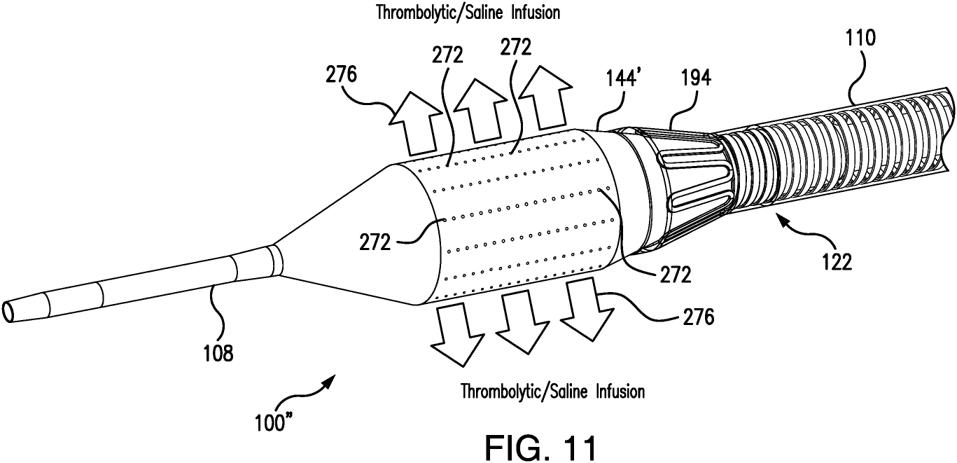

In an additional embodiment of the present guide catheter extension system 100", shown in FIGS. 10A-10B and 11, the balloon member 144' at the inner catheter 108 is fabricated from a balloon material which is loaded with tungsten, and/or barium, gold, or other radiopaque materail. A plurality of micro pores 272 are formed within the balloon material. The micro pores 272 may be of any shape, but, as an example, the micro pores 272 may have the circular configuration. The micro pores 272 may be fabricated by any appropriate method, including, for example, by laser cutting through the balloon material.

The radiopaque materail loading of the balloon material provides that the balloon member 144' is radiopaque without having to insert a contrast into the balloon during inflating.

As required by a surgery, the balloon 144' may be coupled to a reservoir 274 containing a medicinal fluid 276 necessary for the procedure. When the balloon 144' is delivered (in its deflated mode) to the target location within the blood vessel of interest, the balloon 144' is inflated by controllably filling with the medicinal fluid 276 from the reservoir 274. The medicinal fluid 276 subsequently will leak from the balloon 144' through the micro pores 272, as shown in FIG. 11, with a controlled velocity, for example, in the range of one ML per minute at six atmospheric pressures. The inflation of the balloon member 144' results on the opening of the scaffold member 194 to create a funnel like opening 211 as presented in the previous paragraphs.

The system 100" may be used to infuse low dose of thrombolytic therapy with a drug such as, for example, urokinase streptokinase or Tenecteplase, to soften up or break up a thrombus (clot) in a pulmonary artery (or other blood vessel) prior to removal. The delivery of the low dose of the thrombolytic therapy is followed by the balloon member deflation and subsequent removal of the balloon 144', followed by thrombectomy suction (as shown in FIGS. 8A-8E) to remove the weakened thrombus 254 through the funnel like opening 211 of the scaffold member 194 at the distal end 122 of the outer catheter 110.

The balloon member 144' is capable of maintaining a pressure sufficient to inflate and expand the balloon member 144' and expanding the scaffold member 194 at the distal end 122 of the outer catheter 110, using an inflation device 278 held at 6 atm (or other prescribed pressure) to deliver a prescribed flow of the medicinal liquid through the micro pores 272 in the central section of the balloon catheter.

Although this invention has been described in connection with specific forms and embodiments thereof, it will be appreciated that various modifications other than those discussed above may be resorted to without departing from the spirit or scope of the invention, as defined in the appended Claims. For example, functionally equivalent elements may be substituted for those specifically shown and described, certain features may be used independently of other features, and in certain cases, particular locations of elements, steps, or processes, may be reversed or interposed, all without departing from the spirit or scope of the invention, as defined in the appended Claims.

What is claimed is:

1. An intravascular guide catheter extension system for reverse controlled antegrade and retrograde tracking (CART) procedure for chronic total occlusion (CTO) in a blood vessel of interest, the intravascular guide catheter extension system comprising:
a proximal portion, a distal portion, and a middle portion positioned between said proximal and middle portions,
an outer member formed by a flexible substantially cylindrically contoured elongated outer sheath displaceable internally along a guide catheter and defining an outer sheath lumen, said outer sheath having a proximal end and a distal end, wherein said outer sheath extends between said middle portion and distal portion of said intravascular guide catheter extension system, said outer sheath being configured with an outer tip at said distal end and a radially expandable scaffold member positioned at said outer tip at the distal end of said outer sheath, wherein said radially expendable scaffold member is configured with an elongated member shaped into a zig-zag configuration to form a plurality of wing members extending longitudinally along said outer sheath and disposed circumferentially around a longitudinal axis of said outer sheath, said plurality of wing members defining a distal opening and a proximal opening of said radially expandable scaffold member, said radially expandable scaffold member assuming a closed configuration and an opened configuration,
wherein in said closed configuration, said wing members of said radially expandable scaffold member are arranged in a cylindrical formation,
and wherein in said opened configuration, said scaffold member is deformed to displace said wing members to expand said distal opening;
an inner member having an elongated body defining an internal channel extending along a longitudinal axis thereof, said inner member extending internally along said outer sheath lumen of said outer sheath of said outer member in a controllable relationship with said outer sheath, wherein said inner member has a tapered distal tip configured with a tapered delivery micro-catheter having an elongated body of a predetermined length, said tapered delivery micro-catheter being displaceable along a guide wire beyond said distal end of said outer sheath;
a balloon member having a distal section and a proximal section and attached to said tapered distal tip of said inner member at said distal and proximal sections, wherein said proximal section of said balloon member is positioned internally of said radially expandable scaffold member located at said distal end of said outer sheath; and
an inflation lumen extending inside said inner member between said proximal portion of said intravascular guide catheter extension system and said balloon member to provide a fluid passage between a balloon inflation system and said balloon member, wherein said balloon member intermittently assumes an inflated configuration and a deflated configuration, wherein, when said balloon member is controlled to assume the inflated configuration by actuating the balloon inflation system, said balloon member expands and causes said radially expandable scaffold member to assume said opened configuration thereof.

2. The intravascular guide catheter extension system of claim 1, configured for delivery in the blood vessel of interest from one end thereof, further comprising an additional catheter configured for delivery into the blood vessel of interest from another end thereof, said additional catheter having a proximal end and a distal end, wherein the distal end of said additional catheter is received in said radially expandable scaffold member in said opened configuration, through said expanded distal opening defined by said wing members.

3. The intravascular guide catheter extension system of claim 2, being an antegrade guide catheter extension system, wherein said additional catheter is a retrograde catheter.

4. The intravascular guide catheter extension system of claim 1, wherein said elongated member is fabricated from a material selected from a group consisting of a plastically deformable material, Nitinol, stainless steel, cobalt chromium, plastically deformable alloy, plastic, and a combination thereof.

5. The intravascular guide catheter extension system of claim 1, further comprising an elastic distal sheath disposed at said distal end of said outer sheath, said elastic distal sheath being formed with a tubularly shaped portion and tapered portion disposed in an encircling relationship with said outer tip of said distal end of said outer sheath and said proximal section of said balloon member, wherein said wing members configured with said elongated member are embedded into said tubularly shaped portion of said elastic distal sheath.

6. The intravascular guide catheter extension system of claim 1, wherein each of said wing members has a distal end and a proximal end, wherein distal ends of said plurality of wing members form said distal opening of said radially expandable scaffold member, wherein proximal ends of said plurality of wing members form said proximal opening of said radially expandable scaffold member, and wherein, in said opened configuration of said radially expandable scaffold member, said distal ends of said wing members space apart from one another, thus defining an increased diameter of said expanded distal opening.

7. The intravascular guide catheter extension system of claim 1, further comprising an interconnection mechanism disposed in an operative coupling with said inner and outer members and controllably actuated to operate said guide catheter extension system intermittently in an engaged or disengaged modes of operation, wherein said interconnection mechanism is configured to prevent a displacement of said inner member relative to said outer member.

8. The intravascular guide catheter extension system of claim 7,
wherein, in said engaged mode of operation, said inner and outer members of said guide catheter extension sub-system are engaged for a controllable common displacement along the guide wire,
wherein, in said disengaged mode of operation, said inner and outer members are disengaged for retraction of said inner member from said outer member subsequent to deflation of said balloon member, and
wherein, during and upon the retraction of said inner member from said outer member, said radially expandable scaffold member retain the opened configuration thereof.

9. The intravascular guide catheter extension system of claim 1, wherein, during retraction of said outer member from the guide catheter, said plurality of wing members are plastically compressed inside the guide catheter to allow longitudinal motion of the radially expandable scaffold member inside the guide catheter.

10. The intravascular guide catheter extension system of claim 1, wherein, in said deflated configuration, said balloon member is displaced in the blood vessel of interest, and wherein said balloon member is controllably transformed into said inflated configuration subsequent to being positioned at least in alignment with a site of interest for expanding said distal opening of said radially expandable scaffold member.

11. The intravascular guide catheter extension system of claim 1, wherein said micro-catheter is shaped with an outermost distal end having a sharp edge.

12. The intravascular guide catheter extension system of claim 1, further comprising:
an outer member pusher configured with a flattened portion at a distal end thereof and secured to said proximal end of said outer sheath of said outer member.

13. The intravascular guide catheter extension system of claim 12, wherein said inflation lumen includes:
an inflation lumen hypo-tube coupled, by a proximal end thereof, to the balloon inflation system and configured with a skived portion at a distal end thereof, and
an inflation lumen distal shaft having a proximal end overlapping with said skived portion at the distal end of said inflation lumen hypo tube, and a distal end extending towards said balloon member and coupled thereto in fluidly sealed communication therewith.

14. The intravascular guide catheter extension system of claim 7, wherein said interconnection mechanism is selected from a group consisting of a friction-based unit interfacing an outer surface of said inner member and an inner surface of said outer sheath of said outer member, a snap-fit mechanism being configured with at least one snap-fit post formed at said inner member and extending above an external surface thereof, and a combination thereof.

15. The intravascular guide catheter extension system of claim 1, further including a flat wire helical coil member forming at least a portion of respective walls of a member selected from a group consisting of said outer sheath of said outer member and said micro-catheter, wherein said flat wire helical coil is formed with a material selected from a group comprising Nitinol, a radio-opaque material, and a combination thereof.

16. The intravascular guide catheter extension system of claim 1, further including radio-opaque markers attached to at least a location selected from a group consisting of said distal end of said outer sheath, a distal end of said micro-catheter, said tapered distal tip of said inner member in proximity to said proximal and distal sections of said balloon member, and a combination thereof.

17. The intravascular guide catheter extension system of claim 1, further including a pre-shaped curved portion at the distal portion of said intravascular guide catheter extension system.

18. The intravascular guide catheter extension system of claim 17, wherein said curved portion is prefabricated at the distal end of said outer sheath of said outer member.

19. The intravascular guide catheter extension system of claim 18, wherein at said curved portion said outer sheath angularly deviates from a longitudinal axis of said outer sheath at said proximal end thereof at an angle ranging between 30° and 90°.

20. The intravascular guide catheter extension system of claim 1, wherein said balloon member if formed with a radiopaque material loaded balloon material fabricated with a plurality of micro pores, said radiopaque material being selected from a group including tungsten, barium, gold, and combination thereof.

21. The intravascular guide catheter extension system of claim 19, further comprising a medicinal fluid delivered into the balloon member through said inflation lumen, wherein said medicinal fluid exits from said balloon member through said plurality of micro pores.

22. A method for reverse controlled antegrade and retrograde tracking (CART) procedure for treatment of chronic total occlusion (CTO) in a blood vessel of interest, comprising:
(a) assembling a guide catheter extension system, said guide catheter extension system comprising:
an outer member formed by a flexible substantially cylindrically contoured elongated outer sheath defining an outer sheath lumen having a proximal end and a distal end,
a radially expandable scaffold member positioned at an outer tip at the distal end of the outer sheath, wherein said radially expendable scaffold member is configured with an elongated member shaped into a zig-zag configuration to form a plurality of wing members, each wing member extending longitudinally the outer sheath, and the plurality of wing members being disposed circumferentially along walls of the outer sheath around a longitudinal axis of the outer sheath, wherein the radially expandable scaffold member intermittently assumes a closed configuration and an opened configuration, wherein in the closed configuration, the wing members of the radially expandable scaffold member are arranged in a cylindrical tubular formation having a proximal opening and a distal opening, and wherein in the opened configuration, the scaffold member is plastically deformed to expand the distal ends of the wing members from one another to enlarge the distal opening formed by the distal ends of the wing members of the radially expandable scaffold member,
an inner member having an elongated body defining an internal channel extending along a longitudinal axis thereof, the inner member extending internally along the outer sheath lumen of the outer sheath of the outer member in a controllable relationship with the outer sheath, wherein the inner member has a tapered distal tip configured with a tapered delivery micro-catheter having an elongated body of a predetermined length,
a balloon member having a distal section and a proximal section, the balloon member being attached at its proximal and distal sections to the tapered distal tip of the inner member, wherein the proximal section of the balloon member extends internally of the radially expandable scaffold member at the distal end of the outer sheath, and wherein the balloon member assumes intermittently an inflated configuration and a deflated configuration, wherein, when the balloon member is controlled to assume the inflated configuration by actuating a balloon inflation system, the proximal section of the balloon member expands and causes the radially expandable scaffold member to assume the opened configuration thereof;
(b) forming an antegrade dissection of a blood vessel of interest having a total occlusion to form a subintimal space in proximity to the total occlusion in the blood vessel of interest;
(c) inserting an antegrade guide wire into the subintimal space through the antegrade dissection of the blood vessel of interest;
(d) forming a retrograde dissection of the blood vessel of interest in proximity to the total occlusion and inserting a retrograde guidewire into the subintimal space through the retrograde dissection of the blood vessel of interest,
(e) extending the guide wire catheter extension system over the antegrade guidewire in the subintimal space,
(f) actuating an inflation system to inflate the balloon member, thus plastically deforming the radially expandable scaffold member to transform into the opened configuration thereof;
(g) deflating the balloon member;
(h) retracting the inner member from the outer member; and
(i) inserting a distal end of the retrograde guidewire in the expanded distal opening of the radially expandable scaffold member at the distal end of the outer member, and
(j) entering a retrograde catheter into the expanded distal opening of the radially expandable scaffold member of the outer member, and advancing the retrograde catheter inside and along the outer member beyond the proximal end of the outer sheath.

23. The method of claim 22, further comprising:
subsequent to said step (c), advancing a balloon dilatation catheter having a dilatation balloon over said antegrade guidewire into said subintimal space, and
inflating said dilatation balloon to expand the subintimal space.

24. The method of claim 22, further comprising:
prior to said step (e), inserting a guide catheter in the blood vessel of interest over said antegrade guide wire, and
in said step (e), sliding said guide catheter extension system inside and along the guide catheter.

25. The method of claim 22, further comprising:
subsequent to step (i), retracting said outer member from said guide catheter, wherein said expanded wing members of said radially expandable scaffold member are plastically compressed by walls of said guide catheter during the retraction.

26. The method of claim 22, further comprising:
in said step (a), installing an interconnection mechanism in said guide catheter extension system, said interconnection mechanism being configured to prevent a displacement of said inner member relative to said outer member;
in said step (e), controllably actuating said interconnection mechanism to operate said guide catheter extension system in the engaged mode of operation; and in said step (h), controllably actuating said interconnection mechanism to operate said guide catheter extension system in the disengaged mode of operation;
wherein, in said engaged mode of operation, said inner and outer members of said guide catheter extension system are engaged for a controllable integral displacement in the blood vessel of interest, and
wherein, in said disengaged mode of operation, said inner and outer members are disengaged for a controllable retraction of said inner member from said outer members.

27. The method of claim 26, further comprising:
in said step (e), controlling said interconnection mechanism to establish said engaged mode of operation;
advancing said inner and outer members engaged together along the blood vessel of interest, with said balloon member in the deflated configuration thereof, by pushing said outer member, thus causing said micro-catheter of said inner member to slide along the antegrade guidewire towards the subintimal space until said balloon member attached to said tapered distal tip of said inner member is being brought to alignment with the subintimal space; and
subsequent to said step (g), controlling said interconnection mechanism to switch to said disengaged mode of operation; and in said step (h), withdrawing said inner member from said outer member.

28. The method of claim 22, further comprising:
in said step (j), advancing a retrograde micro-catheter of said retrograde catheter into said expanded distal opening of said radially expandable scaffold member, and
subsequent to said step (j), removing said retrograde guidewire from said outer sheath of the outer member, and advancing said retrograde catheter into and out of said outer sheath lumen of said outer member.

29. The method of claim 28, further comprising:
subsequent to said step (j), performing angioplasty and subsequent coronary stenting for coronary revascularization to reconstruct the occlusion lesion in the blood vessel of interest from a distal true lumen to a proximal true lumen at both sides of the subintimal space.

30. The method of claim 22, further comprising:
in said step (a), forming said elongated member of said radially expandable scaffold member from a material including a plastically deformable alloy or plastic, stainless steel, cobalt chromium, Nitinol, a radiopaque material, and a combination thereof.

31. A method for thrombus removal procedure in a blood vessel of interest, comprising:
(a) assembling a guide catheter extension system, said guide catheter extension system comprising:
an outer member formed by a flexible substantially cylindrically contoured elongated outer sheath defining an outer sheath lumen having a proximal end and a distal end,
a radially expandable scaffold member positioned at an outer tip at the distal end of the outer sheath, wherein said radially expendable scaffold member is configured with a shape memory elongated member shaped into a zig-zag configuration to form a plurality of wing members, each wing member extending longitudinally the outer sheath, and the plurality of wing members being disposed circumferentially along walls of the outer sheath around a longitudinal axis of the outer sheath, wherein the radially expandable scaffold member intermittently assumes a closed configuration and an opened configuration, wherein in the closed configuration, the wing members of the radially expandable scaffold member are arranged in a cylindrical tubular formation having a proximal opening and a distal opening, and wherein in the opened configuration, the scaffold member is plastically deformed to expand the distal ends of the wing members from one another to enlarge the distal opening formed by the distal ends of the wing members of the radially expandable scaffold member,
an inner member having an elongated body defining an internal channel extending along a longitudinal axis thereof, the inner member extending internally along the outer sheath lumen of the outer sheath of the outer member in a controllable relationship with the outer sheath, wherein the inner member has a tapered distal tip configured with a tapered delivery micro-catheter having an elongated body of a predetermined length,
a balloon member having a distal section and a proximal section, the balloon member being attached at its proximal and distal sections to the tapered distal tip of the inner member, wherein the proximal section of the balloon member extends internally of the radially expandable scaffold member at the distal end of the outer sheath, and wherein the balloon member assumes intermittently an inflated configuration and a deflated configuration, wherein, when the balloon member is controlled to assume the inflated configuration by actuating a balloon inflation system, the proximal section of the balloon member expands and causes the radially expandable scaffold member to assume the opened configuration thereof;
(b) inserting a guide wire into the blood vessel of interest;
(c) extending the guide wire catheter extension system over the guide wire in the blood vessel of interest towards a thrombus;
(d) operatively coupling an inflation system to said balloon member and actuating the inflation system to inflate the balloon member, thus plastically deforming the radially expandable scaffold member to transform into the opened configuration thereof;
(e) deflating the balloon member;
(f) retracting the inner member from the outer member;
(g) inserting thrombus in the expanded distal opening of the radially expandable scaffold member at the distal end of the outer member,
(h) operatively coupling an aspiration system to the proximal end of said outer member; and
(i) actuating the aspiration system to remove the thrombus from the vessel of interest through the outer member.

32. The method of claim 31, further comprising:
prior to said step (c), inserting a guide catheter in the blood vessel of interest over said guide wire, and
in said step (c), sliding said guide catheter extension system inside and along the guide catheter.

33. The method of claim 32, further comprising:
subsequent to step (i), retracting said outer member from said guide catheter, wherein said expanded wing members of said radially expandable scaffold member are plastically compressed by walls of said guide catheter during the retraction.

34. The method of claim 31, further comprising:
in said step (a), installing an interconnection mechanism in said guide catheter extension system, said interconnection mechanism being configured to prevent a displacement of said inner member relative to said outer member;
prior to said step (c), controllably actuating said interconnection mechanism to operate said guide catheter extension system in the engaged mode of operation; and prior to said step (f), controllably actuating said interconnection mechanism to operate said guide catheter extension system in the disengaged mode of operation;
wherein, in said engaged mode of operation, said inner and outer members of said guide catheter extension system are engaged for a controllable integral displacement in the blood vessel of interest, and wherein, in said disengaged mode of operation, said inner and outer members are disengaged for a controllable retraction of said inner member from said outer members.

35. The method of claim 31, further comprising:
in said step (a), forming said elongated member of said radially expandable scaffold member from a material including a plastically deformable alloy or plastic, stainless steel, cobalt chromium, Nitinol, a radiopaque material, and a combination thereof.

36. The method of claim 31, further comprising:
in said step (a), forming said balloon member from a balloon material loaded with a radiopaque material, and fabricating a plurality of micro pores in said balloon material, and
in said step (d), inflating said balloon member with a medicinal fluid and creating a pressure inside the balloon member sufficient to expel said medicinal fluid from said balloon member through said plurality of micro pores.

37. The method of claim 36, wherein said medicinal fluid is a thrombolytic agent.

38. The method of claim 36, wherein said radiopaque material is selected from a group including tungsten, barium, gold, and a combination thereof.

39. The method of claim 31, further comprising:
in said step (a), pre-shaping said cylindrically contoured elongated outer sheath of said outer member with a curved portion at said distal end of said cylindrically contoured elongated outer sheath.

40. The method of claim 39, wherein, at said curved portion, said cylindrically contoured elongated outer sheath angularly deviates from a longitudinal axis of said cylindrically contoured elongated outer sheath at said proximal end thereof at an angle ranging between 30° and 90°.
